(19) United States
(12) Patent Application Publication
(10) Pub. No.: US 2021/0196230 A1
(43) Pub. Date: Jul. 1, 2021

(54) POSITION REGISTERED SIDEVIEW ULTRASOUND (US) IMAGER INSERTED INTO BRAIN VIA TROCAR

(71) Applicant: BIOSENSE WEBSTER (ISRAEL) LTD., Yokneam (IL)

(72) Inventor: Assaf Govari, Haifa (IL)

(21) Appl. No.: 16/729,436

(22) Filed: Dec. 29, 2019

(51) Int. Cl. A61B 8/08; A61B 5/06; A61B 17/34

(52) U.S. Cl. CPC A61B 8/0841; A61B 5/062; A61B 2017/3413; A61B 17/3403; A61B 5/065

(57) ABSTRACT

An apparatus includes a medical probe and a trocar. The medical probe includes a distal end configured to be inserted into an organ of a patient, the distal end including a magnetic position sensor and a sideview-looking ultrasound imager. The trocar has a channel for insertion of the probe.

### FIELD OF THE INVENTION

The present invention relates generally to invasive medical probes, and particularly to methods and systems for tracking a medical probe in a patient body.

### BACKGROUND OF THE INVENTION

Techniques for medical image guided probing of an organ of a patient were previously proposed in the patent literature. For example, U.S. Pat. No. 10,117,564 describes equipment and procedures in the field of surgery and/or diagnostics and, more particularly, instruments, assemblies and methods for undertaking surgical and/or diagnostic procedures that involve and/or are in proximity to the brain (e.g., cranial procedures and/or surgery). The disclosed assemblies generally include a handle member and an elongated probe that includes an ultrasound transducer. The assemblies may be used in conjunction with K-wires/guidewires, tubular members (e.g., EVD catheters and/or ventricular drains), endoscopes/cameras, and accessory items such as curettes, probes, knives, suction devices, scissors, cautery units, forceps, grasping devices and the like. Advantageous medical diagnostic and surgical instruments, assemblies and methods are provided for use during a broad variety of clinical applications and procedures (e.g., procedures within the cranium and/or in connection with or in proximity to the brain, spinal surgical procedures, orthopedic applications, minimally invasive surgical procedures, etc.).

As another example, U.S. Pat. No. 7,258,668 describes an ultrasonic probe for microscopic operations where the probe consists mainly of an ultrasonic probe body, an elongated tubular member, and a bent handle member. The ultrasonic probe body has a transducer assembly attached to the distal end of a soft elongated tube that is extended from a connector to be coupled to an ultrasonic observation apparatus, and has a coupler mounted on the tube thereof. The transducer assembly and tube are passed through the elongated tubular member. The handle member is attached to the proximal end of the tubular member and includes a coupling mechanism for use in coupling the coupler to the handle member so that the coupler can be uncoupled freely. The probe may scan a plane orthogonal to a direction of insertion of the probe so as to produce a so-called radial image.

### SUMMARY OF THE INVENTION

An embodiment of the present invention provides an apparatus including a medical probe and a trocar. The medical probe includes a distal end configured to be inserted into an organ of a patient, the distal end including a magnetic position sensor and a sideview-looking ultrasound imager. The trocar has a channel for insertion of the probe.

In some embodiments, the distal end further includes a surgical device. In other embodiments, the distal end further includes a forward-looking camera.

There is additionally provided, in accordance with another embodiment of the present invention, a system including a medical probe, a trocar, and a processor. The medical probe includes a distal end configured to be inserted into an organ of a patient, the distal end including a magnetic position sensor and a sideview-looking ultrasound imager. The trocar has a channel for insertion of the probe. The processor is configured to (a) receive one or more reference medical images of the organ, (b) receive signals indicative of an estimated position of the magnetic position sensor in the organ, and estimate a position of the magnetic position sensor based on the signals, (c) receive an ultrasound signal from the sideview-looking ultrasound imager and generate a respective ultrasound image, (d) based on the estimated position, register the ultrasound image with the reference medical image, and (e) present the registered images to a user.

In some embodiments, the processor is further configured to, based on the registered images, present a path in the organ for advancing the distal end.

In some embodiments, the processor is further configured to correct the reference medical images based on the registered images.

In an embodiment, the processor is further configured to alert a user to a detected discrepancy between the ultrasound image and the reference image.

There is further provided, in accordance with another embodiment of the present invention, a method including using a trocar having a channel for insertion of a probe, inserting into an organ of a patient a medical probe comprising a distal end comprising a magnetic position sensor and a sideview-looking ultrasound imager. A reference medical image of the organ is received in a processor. Signals are received, which are indicative of an estimated position of the magnetic position sensor in the organ, and a position of the magnetic position sensor is estimated based on the signals. An ultrasound signal is received from the sideview-looking ultrasound imager, and a respective ultrasound image is generated. Based on the estimated position, the ultrasound image is registered with the reference medical image. The registered images are presented to a user.

There is further yet provided, in accordance with another embodiment of the present invention, a medical probe, including a distal end configured to be inserted into an organ of a patient, the distal end including a magnetic position sensor and a sideview-looking ultrasound imager.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a brain procedure using a surgical apparatus comprising a trocar and guidewire comprising an ultrasound imager, in accordance with an embodiment of the present invention;

FIG. 2 is a schematic, pictorial illustration of the surgical apparatus applied in the brain procedure of FIG. 1, in accordance with an embodiment of the present invention; and

FIG. 3 is a flow chart that schematically illustrates a method and algorithm for registering an ultrasound image received by the US imager of FIG. 1 with a reference medical image, in accordance with an embodiment of the present invention.

### DETAILED DESCRIPTION OF EMBODIMENTS

### Overview

Some medical procedures require a way to guide a medical probe, such as of a surgical apparatus, to an organ of a patient. For example, brain procedures may require navigating a distal end of a probe inserted into the brain via the nose. During such an invasive procedure on the brain, a surgical tool such as a trocar may be inserted, and a guidewire fitted with a forward-looking camera, to be advanced via the trocar to enable a physician to see and treat elements of the brain, such as, for example, infected tissue. However, the camera cannot image brain elements to the sides of the guidewire, such as sensitive blood vessels.

Embodiments of the present invention that are described hereinafter provide an apparatus comprising a probe having a distal end of a guidewire comprising a location sensor (operative in a magnetic tracking system) and a sideview-looking ultrasound imager. In the context of the present disclosure and in the claims, the term “sideview-looking” refers to an ultrasound imager whose field-of-view contains a direction perpendicular to the longitudinal axis of the probe. In other words, a sideview-looking ultrasound imager acquires images of tissue at a direction 90° relative to the longitudinal axis of the probe. This 90° direction, however, need not necessarily fall at the center of the imager's field-of-view.

The location sensor enables sideview images generated by the imager to be correlated (e.g., registered) with other images of the brain, such as reference images provided by a prior CT scan or MRI scan. The apparatus further includes a trocar having a channel for probe insertion.

A system comprising a probe with a position sensor of a magnetic position-tracking system coupled to a distal end of the probe is described in U.S. patent application Ser. No. 15/859,969, filed Jan. 2, 2018, entitled “Tracking a Rigid Tool in a Patient Body,” which is assigned to the assignee of the present patent application, which prior application is hereby incorporated by reference in its entirety herein into this application as if set forth in full.

In some embodiments, a system is provided which is configured to (a) receive one or more reference medical images of the organ, (b) receive signals indicative of an estimated position of the magnetic position sensor in the organ and estimate a respective position of the sensor, (c) receive ultrasound signals from the sideview-looking ultrasound imager and generate a respective ultrasound image, (d) register the ultrasound image with the reference medical image, based on the estimated position, and (e) present the registered images to a user.

The disclosed techniques enable improved safety and quality of minimally invasive medical procedures by allowing registering real-time US images with reference medical images from another imaging modality.

### System Description

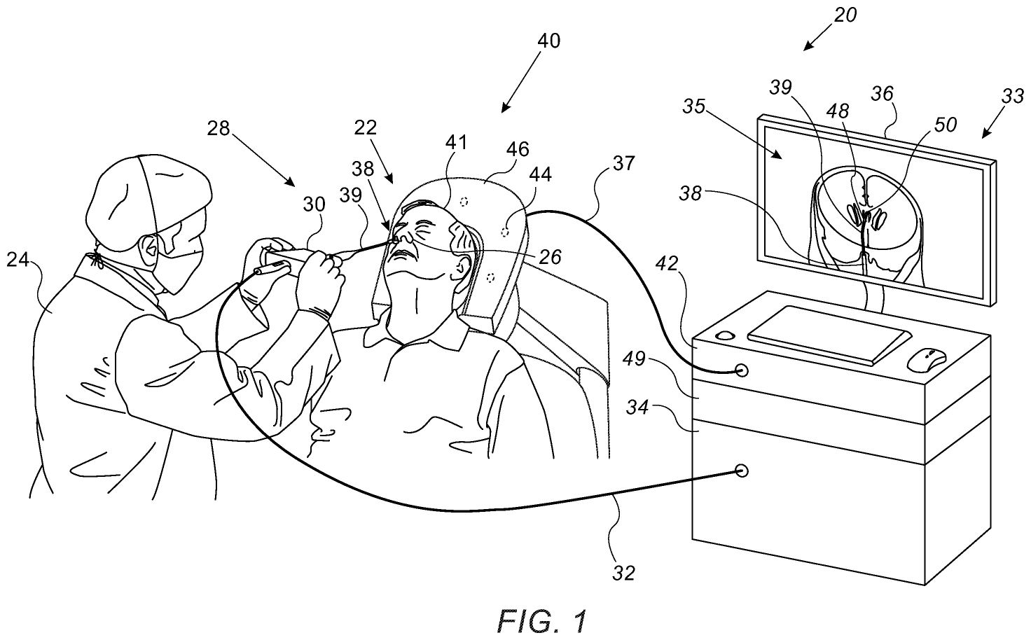

FIG. 1 is a schematic, pictorial illustration of a brain procedure using surgical apparatus 28 comprising a trocar 38 and guidewire 39 comprising an ultrasound imager 50, in accordance with an embodiment of the present invention. In some embodiments, a brain diagnostics and treatment system 20, which comprises surgical apparatus 28, is configured to carry out a brain procedure, such as treating an infection of brain tissue of a patient 22.

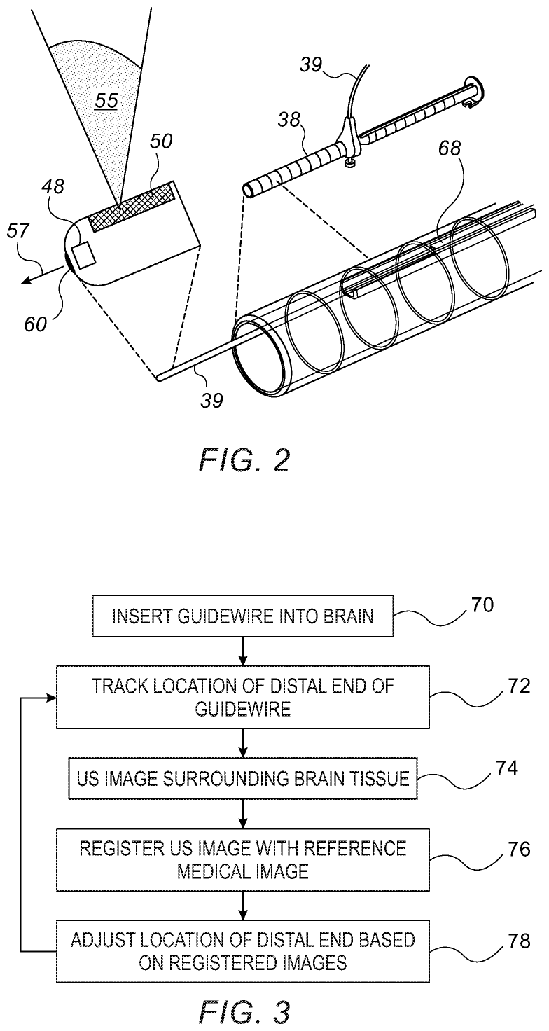

Surgical apparatus 28 comprises guidewire 39 that is inserted into the brain via trocar 38. Guidewire 39 comprises a magnetic position sensor 48, a sideview-looking ultrasound imager 50 and a forward-looking camera (camera shown in FIG. 2), which a physician 24 inserts into a nose 26 of patient 22 to access brain tissue. In the present context, the term “forward-looking” means that the direction of the longitudinal axis of guidewire 39 falls within the field-of-view of the camera.

Surgical apparatus 28 further comprises a handheld proximal-end assembly 30, coupled to a proximal end of trocar 38, which is configured to assist physician 24 to align trocar in a head 41 of patient 22 and subsequently navigate guidewire 39 to target brain tissue.

System 20 comprises a magnetic position-tracking system, which is configured to track a position of sensor 48 in the brain. The magnetic position-tracking system comprises a location pad 40, which comprises field generators 44 fixed on a frame 46. In the exemplary configuration shown in FIG. 1, pad 40 comprises five field generators 44, but may alternatively comprise any other suitable number of generators 44. Pad 40 further comprises a pillow (not shown) placed under head 41 of patient 22, such that generators 44 are located at fixed, known positions external to head 41. The position sensor generates position signals in response to sensing external magnetic fields generated by field generators 44, thereby enabling a processor 34 to estimate the position of sensor 48.

This technique of position sensing is implemented in various medical applications, for example, in the CARTO™ system, produced by Biosense Webster Inc. (Irvine, Calif.) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1, which prior applications are hereby incorporated by reference in their entirety herein into this application as if set forth in full.

In some embodiments, system 20 comprises a console 33, which comprises a memory 49, and a driver circuit 42 configured to drive field generators 44, via a cable 37, with suitable signals so as to generate magnetic fields in a predefined working volume in space around head 41.

Processor 34 is typically a general-purpose computer, with suitable front end and interface circuits for receiving signals from position sensor 48, sideview ultrasound imager 50 and the forward-looking looking camera via a cable 32, and for controlling other components of system 20 described herein.

In some embodiments, processor 34 is configured to register an ultrasound image produced by sideview ultrasound imager 50 with a medical image, such as an MRI image. Processor 34 is able to register the US image by estimating a position of sideview ultrasound imager 50 using position sensor 48. Processor 34 is configured to register the invasive US image and the reference medical image in the coordinate system of the magnetic position-tracking system and/or in a coordinate system of the reference medical image.

In some embodiments, processor 34 is configured to receive, via an interface (not shown), one or more anatomical images, such as reference MRI images depicting two-dimensional (2D) slices of head 41. Processor 34 is configured to select one or more slices from the MRI images, register it with an invasive ultrasound image produced in real time by sideview ultrasound imager 50 to produce a combined image, such as an image 35, and display the selected combined image to physician 24 on user display 36. In the example of FIG. 1, combined image 35 depicts a sectional coronal view of anterior brain tissue of patient 22.

Console 33 further comprises input devices, such as a keyboard and a mouse, for controlling the operation of the console, and a user display 36, which is configured to display the data (e.g., images) received from processor 34 and/or to display inputs inserted by a user using the input devices (e.g., by physician 24).

FIG. 1 shows only elements related to the disclosed techniques, for the sake of simplicity and clarity. System 20 typically comprises additional modules and elements that are not directly related to the disclosed techniques, and thus are intentionally omitted from FIG. 1 and from the corresponding description.

Processor 34 may be programmed in software to carry out the functions that are used by the system, and to store data in memory 49 to be processed or otherwise used by the software. The software may be downloaded to the processor in electronic form, over a network, for example, or it may be provided on non-transitory tangible media, such as optical, magnetic or electronic memory media. Alternatively, some or all of the functions of processor 34 may be carried out by dedicated or programmable digital hardware components. In particular, processor 34 runs a dedicated algorithm as disclosed herein, including in FIG. 3, that enables processor 34 to perform the disclosed steps, as further described below.

### Position Registered Sideview Us Imager Inserted into Brain Via Trocar

FIG. 2 is a schematic, pictorial illustration of surgical apparatus 28 applied in the brain procedure of FIG. 1, in accordance with an embodiment of the present invention. As seen, apparatus 28 includes trocar 38 that includes a channel 68 for guidewire 39. Sideview-looking US imager 50 transmits US energy in the form of a slice section 55, however, in general, imager 50 may produce volumetric US data comprising multiple slices. As seen, slice 55 covers area in a plane perpendicular to a longitudinal direction 57 toward which camera 60 is aimed. Anatomical structures to the side of guidewire 39 may be US imaged, and their detected presence may prompt physician 24 to adjust a path along which to advance guidewire 39. By registering the real-time US image with a reference medical image, such as an MRI image that includes details that are less visible in an US image, the physician may receive critical information that otherwise (i.e., without the registration) may be lacking.

The configuration of surgical apparatus 28 is depicted by way of example for the sake of conceptual clarity. In other embodiments, any alternative configuration may be used, for example, one that employs different types of position sensors, such as those based on electrical impedance signals in addition or alternatively to magnetic signals.

FIG. 3 is a flow chart that schematically illustrates a method and algorithm for registering an ultrasound image received by the sideview-looking US imager 50 of FIG. 1 with a reference medical image, in accordance with an embodiment of the present invention. The process begins when physician 24 inserts trocar 38 through the trocar inserts and advances guidewire 39 into the brain of patient 22, at a guidewire insertion step 70. Next, physician 24 operates system 20 to magnetically track a location in the brain of the distal end of guidewire 39, at a guidewire position tracking step 72.

In an US imaging step 74, as the physician advances guidewire 39, processor 34 generates US slices of brain tissue using echo signals received from sideview-looking US imager 50.

At an image registration step 76, based on the tracked position of US imager 50 (using sensor 48), processor 34 registers the real-time updated US image to a respective reference medical image stored in memory 49, such as from an MRI scan, to produce combined image 35. In an embodiment, processor 34 is further configured to correct the reference medical images based on the registered images, for example, if the treatment removes brain tissue. In another embodiment, the processor is further configured to alert a user to a detected discrepancy between the ultrasound image and the reference image, for example, due to a larger tumor size detected by the US imager, due to tumor growth since the reference image was taken.

Finally, using combined image 35, physician 24 advances guidewire 39 in an informed manner, at a location adjustment step 78, to a target brain tissue, such as an infected tissue. Physician 24 is able to navigate guidewire 39 in such a way by considering the real-time US information on top of the information from the reference medical image, in a combined image 35 provided on display 36. The process then loops back to step 74 to acquire a new US image, so as to generate an updated combined image 35. In an embodiment, processor 34 is further configured to present a path to advance the distal end in the organ on display 36 based on the registered images.

The example flow chart shown in FIG. 3 is chosen purely for the sake of conceptual clarity. In alternative embodiments physician 24 may perform additional steps, such as employing additional monitoring steps (e.g., fluoroscopy) to verify the successful outcome of the procedure, and/or may apply other sensors fitted to distal end 31, for example, to acquire additional clinical data, such as intracranial pressure.

Although the embodiments described herein mainly address brain procedures, the methods and systems described herein can also be used in other applications that require guiding a medical device in other organs, such as located in the abdomen or the chest.

It will thus be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. An apparatus, comprising:
a medical probe, comprising a distal end configured to be inserted into an organ of a patient, the distal end comprising a magnetic position sensor and a sideview-looking ultrasound imager; and
a trocar having a channel for insertion of the probe.

2. The apparatus according to claim 1, wherein the distal end further comprises a surgical device.

3. The apparatus according to claim 1, wherein the distal end further comprises a forward-looking camera.

4. A system, comprising:
a medical probe, comprising a distal end configured to be inserted into an organ of a patient, the distal end comprising a magnetic position sensor and a sideview-looking ultrasound imager;
a trocar having a channel for insertion of the probe; and
a processor, which is configured to:
receive one or more reference medical images of the organ;
receive signals indicative of an estimated position of the magnetic position sensor in the organ, and estimate a position of the magnetic position sensor based on the signals;
receive an ultrasound signal from the sideview-looking ultrasound imager and generate a respective ultrasound image;
based on the estimated position, register the ultrasound image with the reference medical image; and
present the registered images to a user.

5. The system according to claim 4, wherein the processor is further configured to, based on the registered images, present a path in the organ for advancing the distal end.

6. The system according to claim 4, wherein the processor is further configured to correct the reference medical images based on the registered images.

7. The system according to claim 4, wherein the processor is further configured to alert a user to a detected discrepancy between the ultrasound image and the reference image.

8. A method, comprising:
using a trocar having a channel for insertion of a probe, inserting into an organ of a patient a medical probe comprising a distal end comprising a magnetic position sensor and a sideview-looking ultrasound imager; and
in a processor:
receiving a reference medical image of the organ;
receiving signals indicative of an estimated position of the magnetic position sensor in the organ, and estimating a position of the magnetic position sensor based on the signals;
receiving an ultrasound signal from the sideview-looking ultrasound imager, and generating a respective ultrasound image;
based on the estimated position, registering the ultrasound image with the reference medical image; and
presenting the registered images to a user.

9. The method according to claim 8, and comprising, based on the registered images, presenting to a user a path in the organ for advancing the distal end.

10. The method according to claim 8, and comprising correcting the reference medical image based on the registered images.

11. The method according to claim 8, and comprising alerting a user to a detected discrepancy between the ultrasound image and the reference image.

12. A medical probe, comprising a distal end configured to be inserted into an organ of a patient, the distal end comprising a magnetic position sensor and a sideview-looking ultrasound imager.
